(19) United States
(12) Patent Application Publication
(10) Pub. No.: US 2021/0085853 A1
(43) Pub. Date: Mar. 25, 2021

(54) SYSTEMS AND METHODS FOR AUTOMATED ASSESSMENT AND MONITORING OF BODILY FLUIDS

(71) Applicant: University of Washington, Seattle, WA (US)

(72) Inventors: Tony Chen, Seattle, WA (US); Jialu Sun, Seattle, WA (US); Robert Marten Sweet, Seattle, WA (US); John Lawrence Gore, Seattle, WA (US); Jonathan Teng-Chieh Liu, Seattle, WA (US); Jonathan Dov Posner, Seattle, WA (US); Akshay Purushottamji Randad, Seattle, WA (US); Allegra Joyce Branch, Seattle, WA (US); Eleftherios Kampianakis, Seattle, WA (US); Claudia Jimena Covelli Velez, Seattle, WA (US)

(73) Assignee: University of Washington, Seattle, WA (US)

(21) Appl. No.: 16/976,397

(22) PCT Filed: Mar. 1, 2019
(86) PCT No.: PCT/US19/20295

§ 371 Date: Aug. 27, 2020

Related U.S. Application Data
(60) Provisional application No. 62/637,311, filed on Mar. 1, 2018.

(51) Int. Cl. A61M 3/02; A61M 39/22; A61M 25/00

(52) U.S. Cl. CPC A61M 3/0287; A61M 39/22; A61M 25/0041; A61M 2205/3393; A61M 2205/3334; A61M 2205/3313; A61M 2205/18; A61M 2202/0496; A61M 2205/3375

(57) ABSTRACT

Systems and methods for automated assessment and monitoring of bodily fluids are disclosed. In one embodiment, a system for provisioning a treatment composition to a bladder of a patient includes: an irrigation bag that carries the treatment composition; a catheter configured for connecting the irrigation bag to the bladder; a flow regulator configured for controlling a flow of the treatment composition from the irrigation bag to the bladder; and a drainage bag in fluid communication with the bladder through a drainage tubing of the catheter. The drainage tubing is configured for draining urine from the bladder. The system also includes: a first sensor for detecting a first amount of the treatment composition in the irrigation bag; a second sensor for detecting a second amount of urine in the drainage bag; a third sensor for detecting a concentration of particles in the urine carried by the drainage bag; and a controller.

### CROSS-REFERENCE(S) TO RELATED APPLICATION(S)

This application claims the benefit of Provisional Application No. 62/637,311, filed Mar. 1, 2018, which is incorporated herein by reference.

### BACKGROUND

Hematuria is a condition characterized by the increase of blood concentration in a patient's urine. When urine is visibly blood-tinged to the naked eye, the patient is diagnosed with gross hematuria. Hematuria may occur after some surgeries that cause trauma to the urologic system, thereby causing blood to enter the bladder. The condition is most commonly seen in patients that have undergone transurethral resection of the prostate (TURP). Over 150,000 men per year undergo the TURP. Other surgeries, such as kidney stone removal, removal of bladder cancers, and kidney diseases, and urinary tract infections can also cause hematuria.

Hematuria must be treated to prevent clot formation that blocks the urinary tract and prevents the patient from urinating. In general, blood clots increase the risk of dangerous complications during surgery and recovery. Typical treatment includes a continuous bladder irrigation (CBI) process, whereby a nurse flushes saline through the bladder to dilute and remove hematuria. During the CBI, a triple-lumen catheter is inserted into the patient's bladder, through which a nurse can flush saline in and out of the bladder to wash residual blood or small clots. Unfortunately, this procedure requires a frequent process monitoring by the nurse to ensure that blood clots do not form in the bladder. In many settings, the nurse manually adjusts flow rate of the saline every 15 minutes, which is a burdensome task, especially when attending to multiple patients at the same time.

FIG. 1 is a partially schematic view of a continuous bladder irrigation system in accordance with prior art. One of the ports of a triple-lumen catheter 30 is connected through a transparent tube 31 to an irrigation bag 10 that contains saline 11. Saline 11 flows through a drip chamber 15, and further into a bladder 25 (flow path A31). With the illustrated conventional technology, the nurse controls flow rate of saline 11 through an adjustable clamp 20. Saline that is provided to the bladder dilutes hematuria and/or blood clots in urine. Next, urine flows from the bladder to a drainage bag 40 through a drainage tubing 32 (flow path A32) for collection and disposal. The triple-lumen catheter 30 is held inside the bladder by an inflatable balloon 34. Once inflated through a tubing 33, the inflatable balloon 34 prevents the triple-lumen catheter 30 from falling outside of the bladder.

Generally, the nurse relies on the color of the urine in the drainage bag 42 to increase or reduce the flow of the saline through the clamp 20. When the urine 45 is darker, the nurse increases the flow of the saline through the clamp 20, and, vice versa, when the urine 45 is brighter the nurse decreases the flow of the saline through the clamp 20.

FIG. 2 is an isometric view of a triple-lumen catheter 30 in accordance with prior art. The triple-lumen catheter 30 includes tubes 31, 32 and 33. As explained above, tubes 31 and 32 are respectively dedicated to irrigation and drainage of hematuria. The irrigation and drainage are performed through lumens 31-L and 32-L, respectively. The third tube, tube 33, is connected to the inflatable balloon 34 through a lumen 33-L.

However, the nurse typically attends to multiple patients at the same time, therefore frequently not being able to make timely adjustments of the saline flow for all the patients. Furthermore, the nurse relies on his/her subjective assessment of the amount of the hematuria in the urine, therefore reducing the predictability of the treatment. For instance, in many situations, different nurses may dial-in different flows of saline based on the same objective indicia of a patient's condition. Moreover, accumulation of urine in the drainage bag represents a time average of the flow into the bag, which does not necessarily correspond to the contemporaneous condition (e.g., concentration of blood) in the urine that is in the bladder. Accordingly, there remains a need for treatment systems that improve accuracy and predictability, while reducing cost of the hematuria treatment.

### SUMMARY

This summary is provided to introduce a selection of concepts in a simplified form that are further described below in the Detailed Description. This summary is not intended to identify key features of the claimed subject matter.

Briefly, the inventive technology is directed to flushing the hematuria, blood clots and/or solid particles from the bodily organs, for example, from the bladder. In different embodiments, the bodily fluids may be generated in brain, peritoneal cavity, stomach, chest cavity including pleura, urostomy, or non-anatomic fluid collections such as abscesses or lymphoceles, and can include particles from blood, lymph, radiotracers, tumor markers, pharmaceutical metabolites, dyes, and luminescent particles. In the context of the inventive technology, the term “particles” refers to particles that either remain solid or dissolve in a bodily fluid. Furthermore, for brevity and conciseness, the disclosure below refers to the blood particles being carried by the urine, but other embodiments may include other bodily fluids and particles.

In some embodiments, the flow of saline (or other diluting substance) from the irrigation bag to bladder may be regulated by an automated valve. The control signal to the valve may be based on the measured weight of the irrigation bag that is compared to weight of the drainage bag with the urine. If the weight of the drainage bag does not increase approximately as fast as the weight of the irrigation bag decreases, such an imbalance may indicate that the bladder is blocked by a blood clot or other obstruction. In response to this imbalance, the controller may partially or completely close the valve on the irrigation line to balance the flow and may also set an alarm signal for a nurse to check the status of the patient.

In some embodiments, the inventive technology includes a color detector for detecting a concentration of blood (or other particles) in urine (or other bodily fluid). If the color detector detects a relatively high concentration of blood in urine, the controller may open the valve of the irrigation catheter to increase the flow of saline. Conversely, if the color detector detects a relatively low concentration of blood in urine, the controller may close the valve on the irrigation catheter to decrease the flow of saline.

In some embodiments, the color detector (or other sensor) may be configured proximately to a bend of the drainage tubing. In operation, the tubing may be held in a housing to limit the amount of the outside stray light and to secure the drainage tubing into a target shape of the bend. The housing may also carry a source of light and control electronics. In many embodiments, securely holding the bend of the drainage tubing at a prescribed distance from the color sensor improves the accuracy and repeatability of the measurements, because unsecured tubing may oscillate or move around, thus resulting in less accurate and repeat able measurements. Furthermore, a relatively small volume of urine kept in the bend of the drainage tubing more closely approximates the contemporaneous concentration of blood in the urine that is in the bladder.

In one embodiment, a system for provisioning a treatment composition to a bladder of a patient includes: an irrigation bag that carries the treatment composition; a catheter configured for connecting the irrigation bag to the bladder; a flow regulator configured for controlling a flow of the treatment composition from the irrigation bag to the bladder; and a drainage bag in fluid communication with the bladder through a drainage tubing of the catheter. The drainage tubing is configured for draining urine from the bladder. The system also includes: a first sensor for detecting a first amount of the treatment composition in the irrigation bag; a second sensor for detecting a second amount of urine in the drainage bag; and a third sensor for detecting a concentration of particles in the urine carried by the drainage bag. The system also includes a controller configured for: receiving data representative of the first amount of the treatment composition in the irrigation bag and the second amount of urine in the drainage bag; receiving data representative of the concentration of particles in the urine carried by the drainage bag; and based on received data, controlling the flow regulator.

In one aspect, the particles in the urine are blood particles. In another aspect, the treatment composition is configured to dilute hematuria or clot in the bladder. In one aspect, the first sensor is a weight sensor, a level sensor or a volume sensor. In one aspect, the second sensor is a weight sensor, a level sensor or a volume sensor. In one aspect, the third sensor is a color sensor, a wavelength sensor or an ultrasound sensor.

In one aspect, a hematuria measurement system is configured to at least partially carry the third sensor and the drainage tubing of the catheter, wherein the hematuria measurement system holds the drainage tubing in a bent position configured to form a pool of urine within a bend of the drainage tubing. In another aspect, the bend of the drainage tubing is U-shaped or W-shaped. In one aspect, the third sensor is a color sensor configured to differentiate among shades of the pool of urine within the bend of the drainage tubing.

In one aspect, the flow regulator includes: a motorized valve; and a motor configured to operate the motorized valve, where the motor is in a data communication with the controller. In another aspect, the motorized valve includes a motorized clamp.

In one aspect, the treatment composition comprises saline. In another aspect, the treatment composition comprises medication.

In one embodiment, a method for delivering a treatment composition to a bladder of a patient includes: detecting a first amount of the treatment composition in an irrigation bag by a first sensor; flowing the treatment composition from the irrigation bag to the bladder through a catheter; controlling a flow of the treatment composition by a flow regulator; detecting a second amount of urine in a drainage bag by a second sensor; comparing, by a controller, the first amount of the treatment composition in the irrigation bag and the second amount of urine in the drainage bag; determining, by the controller, whether the flow of the treatment composition from the irrigation bag is balanced by a flow of urine from the bladder; and sending, by the controller, a control signal to the flow regulator of the treatment composition.

In one aspect, the method also includes setting an alert signal when the flow of the treatment composition from the irrigation bag is not balanced by the flow of urine from the bladder. In another aspect, the method also includes: detecting, by a hematuria measurement system, a concentration of blood in the urine carried by the drainage bag; and based on detecting by the hematuria measurement system, sending the control signal to the flow regulator by the controller.

In one aspect, the method includes setting an alert signal when the concentration of blood in the urine carried by the drainage bag exceeds a predetermined threshold. In one aspect, the hematuria measurement system at least partially holds the catheter in a bent position that enables forming a pool of urine within a bend of the catheter. In another aspect, the bend of the drainage tubing is U-shaped or W-shaped.

In one aspect, the hematuria measurement system includes a color sensor configured to differentiate among shades of the pool of urine within the bend of the catheter. In one aspect, the treatment composition is configured to dilute hematuria or clot in the bladder.

In one embodiment, a device for determining a particle concentration in a bodily fluid includes: a housing; a drainage tubing attached to the housing, where the drainage tubing includes at least one bend configured for holding the bodily fluid; and a color sensor configured for detecting the particle concentration in the bodily fluid held in the at least one bend of the drainage tubing.

In another aspect, a color sensor control circuit is configured to: control the color sensor; and receive data from the color sensor.

In one aspect, the system also includes a tube holder configured to, in operation, at least partially surround the drainage tubing and the color sensor. In another aspect, the housing is clam-shell type housing, and the tube holder at least partially surrounds the drainage tubing and the color sensor when the housing is closed. In another aspect, the housing also includes a source of light configured to illuminate the at least one bend of the drainage tubing. In one aspect, the source of light is configured inside the tube holder when the housing is closed.

In one aspect, the device also includes: a drainage bag weight sensor configured to detect a weight of a drainage bag; and a weight sensor control circuit configured to receive data from the weight sensor. In another aspect, the drainage bag weight sensor and the weight sensor control circuit are configured inside the housing.

In one aspect, the bodily fluid comprises urine and particles in the bodily fluid comprise blood particles.

In one embodiment, a method for determining a particle concentration in a bodily fluid includes: flowing the bodily fluid through a drainage tubing, where the drainage tubing is shaped to include at least one bend inside a housing; and detecting, by a color sensor, the particle concentration in the bodily fluid held in the at least one bend of the drainage tubing.

In one aspect, the method includes: flowing the bodily fluid through the at least one bend into a drainage bag; and detecting, by a weight sensor, a weight of the drainage bag. In another aspect, the weight sensor is configured at least partially inside the housing.

In one aspect, the method also includes at least partially occluding the at least one bend by a tube holder. In another aspect, at least one bend is occluded by a tube holder when the housing is closed. In one aspect, the method also includes illuminating the at least one bend of the drainage tubing by a source of light. In another aspect, the source of light is configured inside a tube holder when the at least one bend is occluded by a tube holder.

### DESCRIPTION OF THE DRAWINGS

The foregoing aspects and many of the attendant advantages of the inventive technology will become more readily appreciated as the same are understood with reference to the following detailed description, when taken in conjunction with the accompanying drawings, wherein:

FIG. 1 is a partially schematic view of a continuous bladder irrigation system in accordance with prior art;

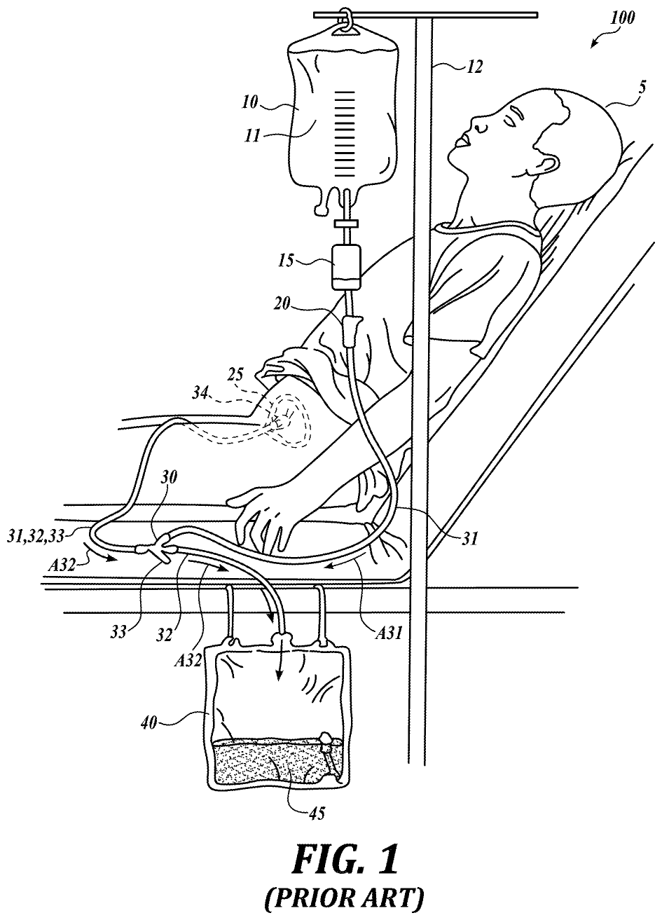

FIG. 2 is an isometric view of a triple-lumen catheter in accordance with prior art;

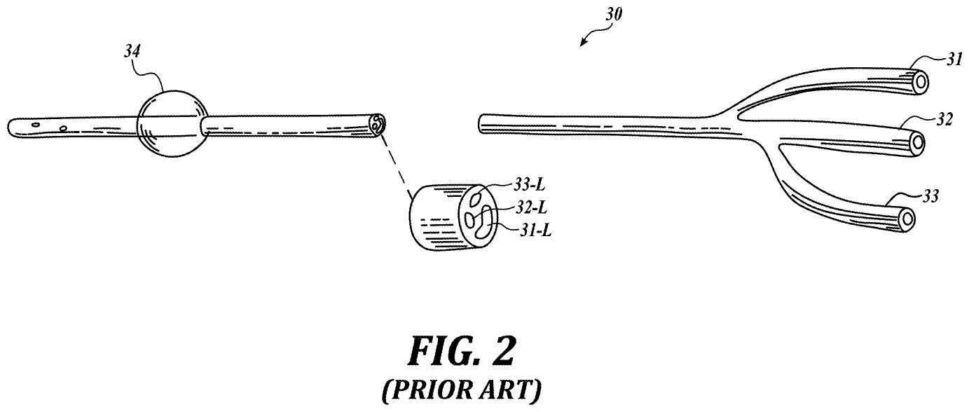

FIG. 3 is a partially schematic view of a continuous bladder irrigation system in accordance with an embodiment of the present technology;

FIG. 4 is a plan view of a hematuria measurement system in accordance with an embodiment of the present technology;

FIG. 5A is a partially schematic view of hematuria specimens in accordance with embodiments of the present technology;

FIG. 5B is a schematic view of a urine retention mechanism in accordance with an embodiment of the present technology;

FIG. 6 is an isometric view of a flow regulator in accordance with embodiments of the present technology;

FIG. 7 is a flowchart of a control process in accordance with an embodiment of the present technology;

FIG. 8 is a graph of drip rate, drainage bag weight and irrigation bag obtained in accordance with an embodiment of the present technology; and

FIG. 9 is a graph of hematuria level obtained in accordance with an embodiment of the present technology.

### DETAILED DESCRIPTION

While several embodiments have been illustrated and described, it will be appreciated that various changes can be made therein without departing from the spirit and scope of the claimed subject matter.

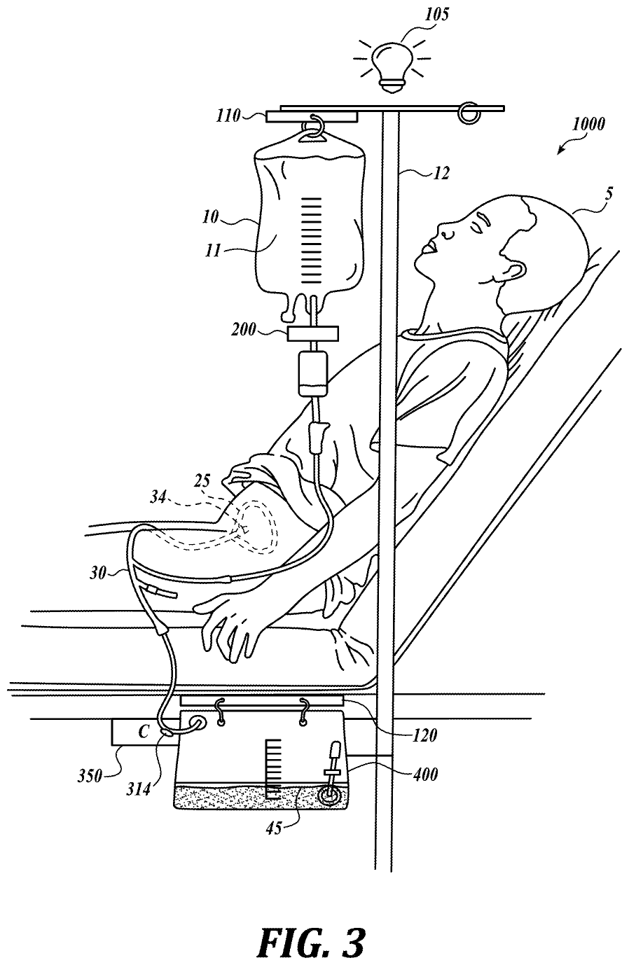

FIG. 3 is a partially schematic view of a continuous bladder irrigation system 1000 in accordance with an embodiment of the present technology. In some embodiments, the irrigation bag 10 may be carried by a weight sensor 110. The illustrated irrigation bag 10 includes saline 11, but in different embodiments the irrigation bag may include other liquids, for example, water mixed with other components like sugar (e.g., Dextrose or Glucose), water mixed with antibiotics, painkillers or other medications. Furthermore, a combination of urine and blood is referred to when describing the inventive technology, however, different combinations of bodily fluids and inorganic or organic particles are also encompassed by the embodiments of the present technology.

In operation, saline 11 drains through the catheter 30 into the bladder 25. The hematuria and/or blood clots inside the bladder are diluted and flown into a drainage bag 400 as a mixture of urine and blood 45.

In some embodiments, a weight sensor 120 supports the drainage bag 400. In some embodiments, a controller 350 receives data from the weight sensors 110 and 120. Based on the received data, the controller 350 detects whether weight reduction of the irrigation bag 10 corresponds (or at least approximately corresponds) to weight increase of the drainage bag 400. If, for example, the weight reduction of the irrigation bag 10 exceeds weight increase of the drainage bag 400, an excessive accumulation of the saline 11 in the patient's bladder 25 may occur. In response, the controller 350 may issue a control command to a flow regulator 200 (e.g., a valve or a clamp) to reduce or stop the flow of saline 11 into the bladder 25. Conversely, when the weight reduction of the irrigation bag 10 is smaller than the weight increase of the drainage bag 400, the system may not be providing enough saline to the bladder, and the controller 350 may issue control command to the flow regulator 200 to increase the flow of saline 11. In different embodiments, the control of the flow of saline 11 may be based upon, for example, a flow meter (not shown) configured to measure the flow of saline.

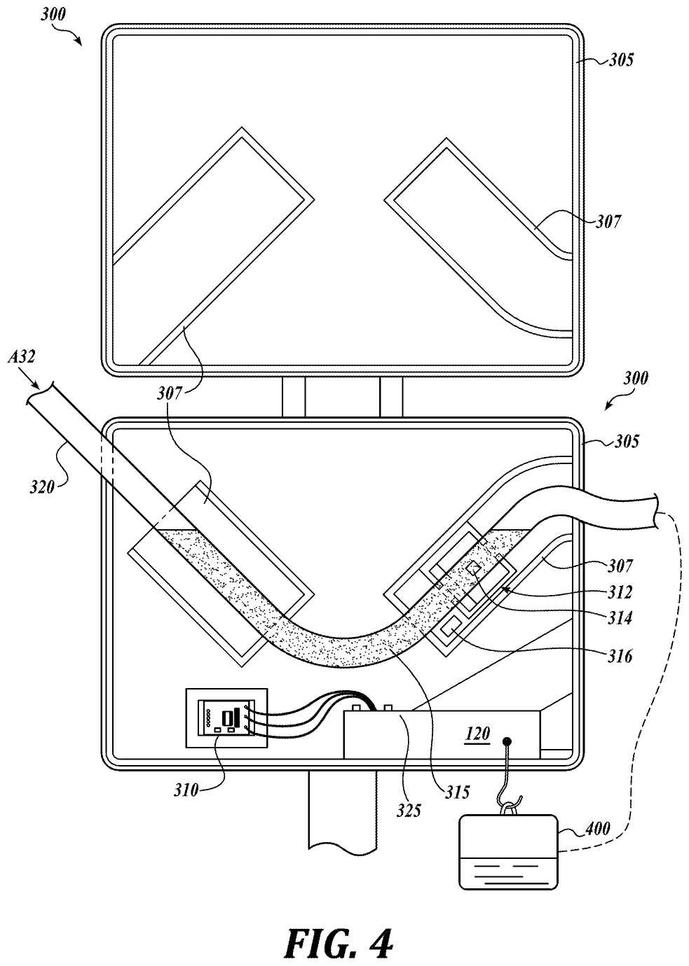

In some embodiments, a sensor 314 (e.g., a color sensor, a wavelength sensor, an ultrasound sensor) may detect color of urine 45 as it flows through a transparent or semitransparent catheter 30 toward the drainage bag 400. Based on the signal received from the color sensor 314, the controller 350 may regulate the flow of saline through the flow regulator 200 or set an alert signal 105 to, for example, alert the nurse about unusual or dangerous conditions. Some embodiments of the color sensor 314 and the accompanying electrical and mechanical hardware are described with respect to FIG. 4 below.

FIG. 4 is a plan view of a hematuria measurement system 300 in accordance with an embodiment of the present technology. The illustrated embodiment includes a housing 305 having tube holders 307 that keep the drainage tubing 320 in a shape configured to retain a urine pool 315 inside the bend of the tubing. In some embodiments, the housing 305 may be configured as clamshell housing. When the housing 305 is closed, the tube holders 307 secure the drainage tubing 320 in its prescribed shape.

In some embodiments, the color sensor 314 is held proximately to the urine pool 315 in a relatively fixed position that facilitates repeatable measurements of the particle concentration in the urine pool. The color sensor 314 may be controlled by a controller 312. In some embodiments, a source of light 316 may illuminate the drainage tubing 320 to facilitate operation of the color sensor 314. In operation, closing of the housing 305 prevents or at least minimizes irradiance of stray light on the color sensor 314.

In some embodiments, the hematuria measurement system 300 includes the weight sensor 120 that can measure the weight of urine in the drainage bag 400. A controller 310 may control the weight sensor 120. In some embodiments, a power source 325 provides power to the sensors (e.g., light sensor and/or weight sensor) and the controllers 310 and 312 of the hematuria measurement system 300.

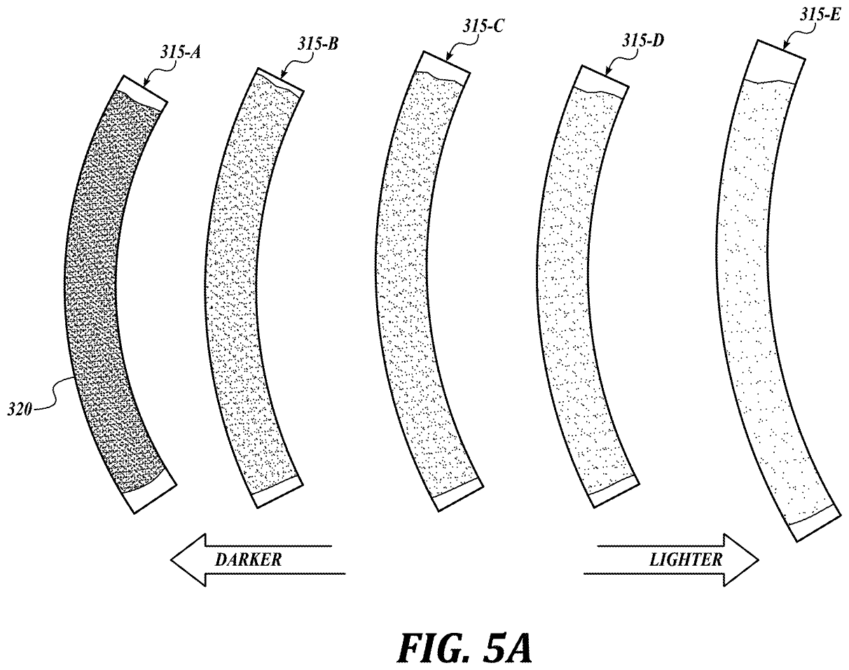

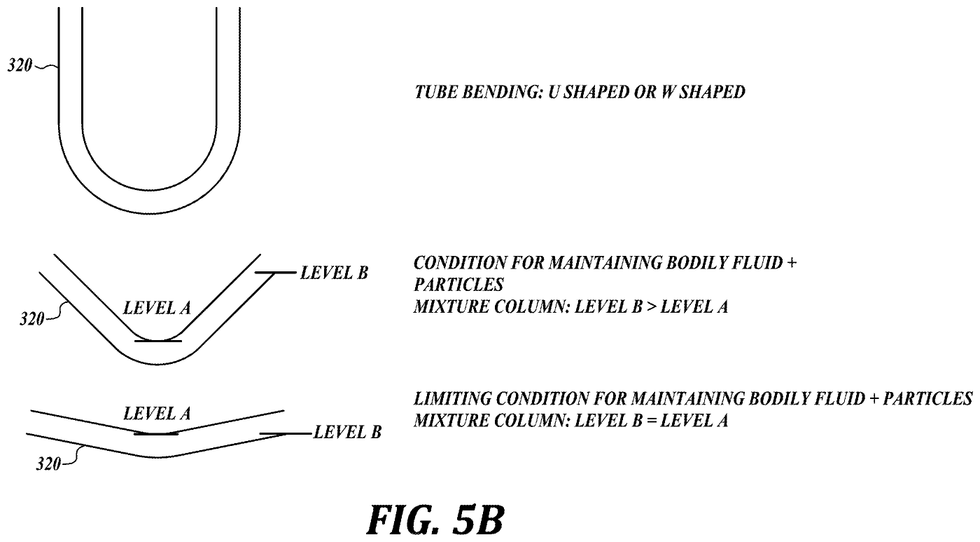

FIG. 5A is a partially schematic view of hematuria specimens in accordance with embodiments of the present technology. The illustrated specimens 315-A to 315-E represent urine (or other bodily fluid) with differing concentrations of blood (or other particles). In operation, a color sensor (not shown) can distinguish between the illustrated concentrations. In many applications, it may be enough for the color sensor to distinguish among several (e.g., 5 or so) differing shades of urine without having to determine the exact numerical concentration of the particles in the bodily fluid. For the controller to, for example, regulate the opening of the control valve, set an alarm signal, or set other actuators or alarms, it may be enough to recognize that the concentration of the particles in the bodily fluid is below or above the level shown by, for example, the specimen 315-C.

FIG. 5B is a schematic view of a urine retention mechanism in accordance with an embodiment of the present technology. The drainage tubing 320 is shown in three representative shapes. The upper configuration of the drainage tubing 320 is a U-shaped tube. In other embodiments, the drainage tubing 320 may have a W-shaped bend, providing two spaces for retaining the bodily fluid.

The middle configuration of the drainage tubing 320 in FIG. 5B illustrates a condition for maintaining a pool of bodily fluid with particles. Here, level B is above level A, thus providing for a pool of bodily fluid that extends up to the level B.

The lower configuration of the drainage tubing 320 illustrates a limiting condition for maintaining a pool of bodily fluid. Under this limiting condition, level A corresponds to level B. In some embodiments, this limiting condition determines a minimum relative height of the level B with respect to the level A that still retains a pool of bodily fluid.

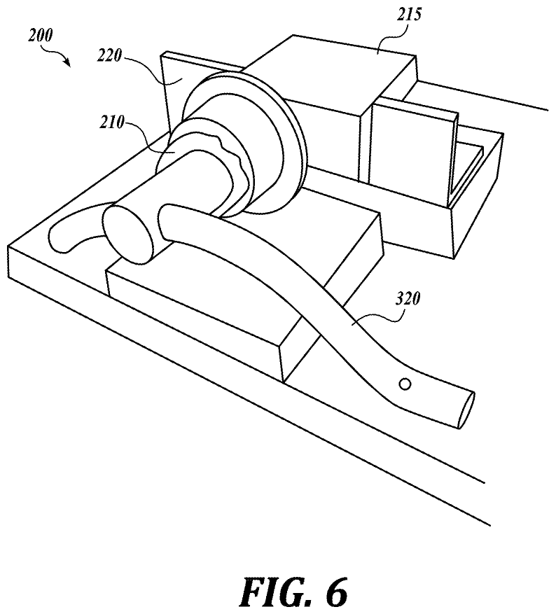

FIG. 6 is an isometric view of a flow regulator 200 in accordance with embodiments of the present technology. In some embodiments, the flow regulator 200 includes a pinch valve 210 the pinches the drainage tubing 322 to reduce the flow of urine through the tubing. In other embodiments, the valve 210 may be based on a lever arm, a roller clamp, a T-handle, or other mechanisms. In some embodiments, the valve 210 may be driven by a motor 215 that is secured by a mounting flange 220, but other securements of the motor 215 are also possible. In operation, the motor 215 may be controlled by the controller 350, as explained in more detail with respect to FIG. 3 above.

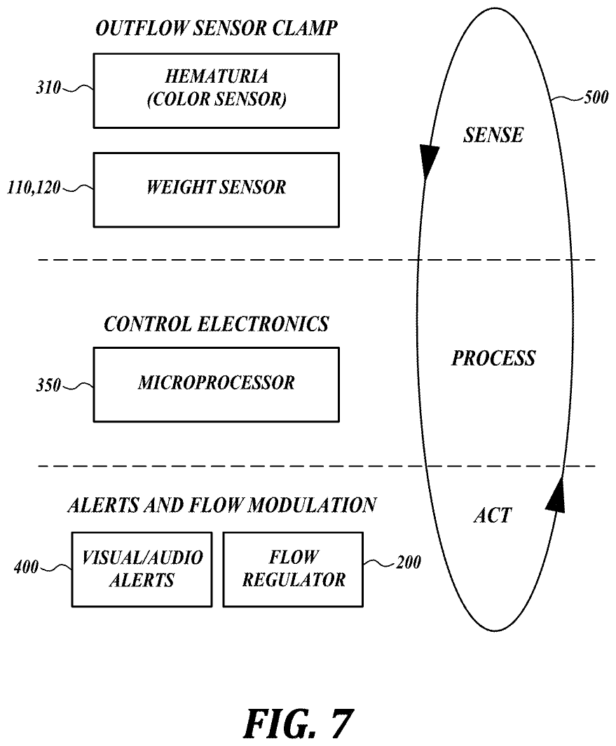

FIG. 7 is a flowchart of a control process in accordance with an embodiment of the present technology. In some embodiments, the control process includes collecting data from the weight sensors 110, 120 and/or color sensor 310 by the microprocessor 350. Based on this input, the microprocessor 350 may issue visual or audio alerts 400, and may control the flow regulator 200 (e.g., a valve). The microprocessor 350 may include a combination of hardware and software to execute the above tasks. A loop 500 illustrates such sense—process—act control loop.

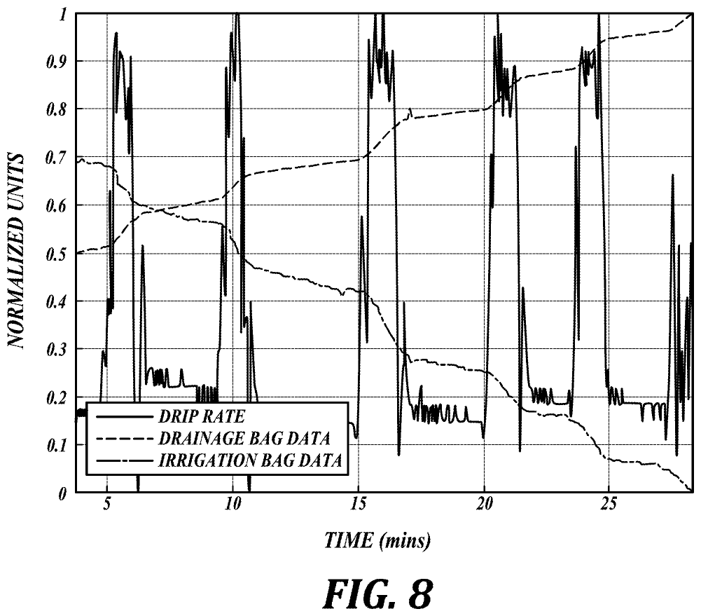

FIG. 8 is a graph of drip rate, drainage bag weight and irrigation bag weight obtained in accordance with an embodiment of the present technology. The vertical axis represents normalized units (e.g., flow rate or normalized weight). The horizontal axis represents time in minutes. Dash line denotes weight of the drainage bag and dash-dot line denotes weight of the irrigation bag. Solid line represents a flow rate of the fluid (e.g., saline) out of the irrigation bag. The illustrated results were obtained by the embodiments of the present technology.

Over the time of about 30 minutes, the irrigation bag loses weight, because it provides saline to the bladder of the patient, while the drainage bag gains weight, because it receives urine from the bladder of the patient. A relatively slow decrease of the weight of the irrigation bag (marked by a relatively gradual slope of the dash-dot line) corresponds to the similarly slow increase of the weight of the drainage bag, whereas a relatively fast decrease of the weight of the irrigation bag (marked by a relatively steep slope of the dash-dot line) corresponds to the similarly fast increase of the weight of the drainage bag. Correspondingly, the periods of relatively high drip rate (solid line) correspond to the periods of the relatively fast decrease of the weight of the irrigation bag, whereas the periods of relatively slow drip rate correspond to the periods of the relatively slow decrease of the weight of the irrigation bag. The above correspondence generally indicates absence of the clot-induced blockage of the urine outflow of the patient. In some embodiments, the scenario shown in FIG. 8 would not trigger an alarm, but would instead be considered an acceptable operational scenario.

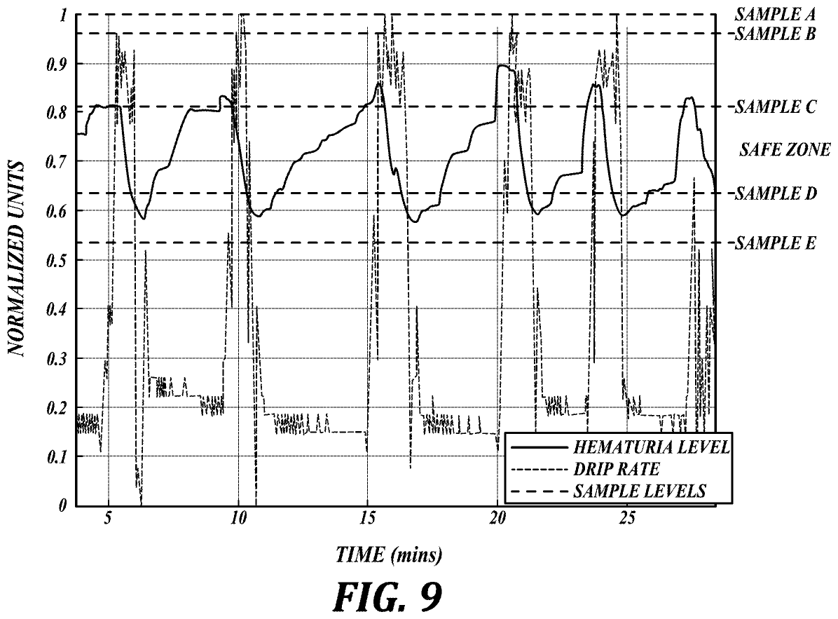

FIG. 9 is a graph of hematuria level obtained in accordance with an embodiment of the present technology. The vertical axis represents normalized units (e.g., flow rate or hematuria level). The horizontal axis represents time in minutes. Solid line represents a hematuria level in the urine that flows out of the patient's bladder through, for example, drainage tubing 320 inside the device 300. Short dash line represents flow rate of the fluid (e.g., saline) out of the irrigation bag. Long dash line represents the color levels corresponding to, for example, samples 315-A to 315-E shown in FIG. 5A. The illustrated results were obtained by the embodiments of the present technology.

In operation, periods of relatively low drip rates of fluid from the irrigation bag (e.g., a relative flatlining of the drip rates in the range of about 0.1 to about 0.3) are typically followed by an increase in the concentration of blood in the urine, as seen by the hematuria levels trending toward sample A and sample B in the graph. In response, the system increases the drip rates of the fluid from the irrigation bag (see, e.g., relatively high values of the drip rates in the range of about 0.8 to about 1), which in turn dilute the hematuria in blood, therefore driving hematuria level back toward the target zone (e.g., between sample C and sample D). Therefore, over most of the operational time the system autonomously maintains hematuria level between the sample C and sample D levels, which is a “safe zone” level.

Many embodiments of the technology described above may take the form of computer- or controller-executable instructions, including routines executed by a programmable computer or controller. Those skilled in the relevant art will appreciate that the technology can be practiced on computer/controller systems other than those shown and described above. The technology can be embodied in a special-purpose computer, controller or data processor that is specifically programmed, configured or constructed to perform one or more of the computer-executable instructions described above. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like).

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The terms “about,” “approximately,” etc., mean plus or minus 5% of the stated value.

From the foregoing, it will be appreciated that specific embodiments of the technology have been described herein for purposes of illustration, but that various modifications may be made without deviating from the disclosure. Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology. Accordingly, the disclosure can encompass other embodiments not expressly shown or described herein.

1. A system for provisioning a treatment composition to a bladder of a patient, the system comprising:
an irrigation bag that carries the treatment composition;
a catheter configured for connecting the irrigation bag to the bladder;
a flow regulator configured for controlling a flow of the treatment composition from the irrigation bag to the bladder;
a drainage bag in fluid communication with the bladder through a drainage tubing of the catheter, wherein the drainage tubing is configured for draining urine from the bladder;
a first sensor for detecting a first amount of treatment composition in the irrigation bag;
a second sensor for detecting a second amount of urine in the drainage bag;
a third sensor for detecting a concentration of particles in urine carried by the drainage bag; and
a controller configured for:
receiving data representative of the first amount of treatment composition in the irrigation bag and the second amount of urine in the drainage bag,
receiving data representative of the concentration of particles in the urine carried by the drainage bag, and
based on received data, controlling the flow regulator.

2. The system of claim 1, wherein the particles in the urine are blood particles and wherein the treatment composition is configured to dilute hematuria or clot in the bladder.

3. (canceled)

4. The system of claim 1, wherein the first sensor and the second sensor are each a weight sensor, a level sensor or a volume sensor, and wherein the third sensor is a color sensor, a wavelength sensor or an ultrasound sensor.

5-6. (canceled)

7. The system of claim 1, further comprising a hematuria measurement system configured to at least partially carry the third sensor and the drainage tubing of the catheter, wherein the hematuria measurement system holds the drainage tubing in a bent position configured to form a pool of urine within a bend of the drainage tubing.

8. The system of claim 7, wherein the bend of the drainage tubing is U-shaped or W-shaped.

9. The system of claim 7, wherein the third sensor is a color sensor configured to differentiate among shades of the pool of urine within the bend of the drainage tubing.

10. The system of claim 1, wherein the flow regulator comprises:
a motorized valve; and
a motor configured to operate the motorized valve, wherein the motor is in a data communication with the controller.

11-13. (canceled)

14. A method for delivering a treatment composition to a bladder of a patient, the method comprising:
detecting a first amount of treatment composition in an irrigation bag by a first sensor;
flowing the treatment composition from the irrigation bag to the bladder through a catheter;
controlling a flow of treatment composition by a flow regulator;
detecting a second amount of urine in a drainage bag by a second sensor;
comparing, by a controller, the first amount of treatment composition in the irrigation bag and the second amount of urine in the drainage bag;
determining, by the controller, whether the flow of treatment composition from the irrigation bag is balanced by a flow of urine from the bladder; and
sending, by the controller, a control signal to the flow regulator.

15. (canceled)

16. The method of claim 14, further comprising:
detecting, by a hematuria measurement system, a concentration of blood in urine carried by the drainage bag;
based on detecting by the hematuria measurement system, sending the control signal to the flow regulator by the controller; and
setting an alert signal when the concentration of blood in urine carried by the drainage bag exceeds a predetermined threshold.

17. (canceled)

18. The method of claim 16, wherein the hematuria measurement system at least partially holds the catheter in a bent position that enables forming a pool of urine within a bend of the catheter.

19. The method of claim 18, wherein the bend of the drainage tubing is U-shaped or W-shaped.

20. The method of claim 18, wherein the hematuria measurement system comprises a color sensor configured to differentiate among shades of the pool of urine within the bend of the catheter.

21. The method of claim 14, wherein the flow regulator comprises:
a motorized valve; and
a motor configured to operate the motorized valve, wherein the motor is in a data communication with the controller.

22. The method of claim 14, wherein the treatment composition is configured to dilute hematuria or clot in the bladder.

23. A device for determining a particle concentration in a bodily fluid, the device comprising:
a housing;
a drainage tubing attached to the housing, wherein the drainage tubing includes at least one bend configured for holding the bodily fluid; and
a color sensor configured for detecting the particle concentration in the bodily fluid held in the at least one bend of the drainage tubing.

24. The device of claim 23, further comprising a color sensor control circuit configured to:
control the color sensor; and
receive data from the color sensor.

25. The device of claim 24, wherein the housing is a clam-shell housing, and wherein the tube holder at least partially surrounds the drainage tubing and the color sensor when the housing is closed, the device further comprising:
a tube holder configured to, in operation, at least partially surround the drainage tubing and the color sensor; and
a source of light configured to illuminate the at least one bend of the drainage tubing, wherein the source of light is configured inside the tube holder when the housing is closed.

26-28. (canceled)

29. The device of claim 23, further comprising:
a weight sensor configured to detect a weight of a drainage bag that is in fluid communication with the drainage tubing; and
a weight sensor control circuit configured to receive data from the weight sensor.

30. The device of claim 29, wherein the drainage bag weight sensor and the weight sensor control circuit are configured inside the housing.

31. The device of claim 23, wherein the bodily fluid comprises urine and wherein particles in the bodily fluid comprise blood particles.

32-38. (canceled)
